(19) United States
(12) Patent Application Publication
(10) Pub. No.: US 2009/0105576 A1
(43) Pub. Date: Apr. 23, 2009

(54) ELECTRODE CONDUCTIVE ELEMENT

(76) Inventors: Nam Hoai Do, New South Wales (AU); Geoffrey Ross Mackellar, Elanora Heights (AU); Lori Ann Washbon, San Francisco, CA (US)

Correspondence Address: FISH & RICHARDSON P.C., PO BOX 1022, MINNEAPOLIS, MN, 55440-1022, US

(21) Appl. No.: 11/876,654

(22) Filed: Oct. 22, 2007

(51) Int. Cl. A61B 5/04

(52) U.S. Cl. 600/382

(57) ABSTRACT

An apparatus and technique for sensing biopotential signals wherein a conductive element is formed from a non-adhesive hydrogel material and configured to provide a conductive path between an electrode and a subject's skin for transmitting EEG signals from the subject to the electrode.

### TECHNICAL FIELD

This invention relates to an apparatus that can be used for bio-sensing.

### BACKGROUND

An electrode system to capture bioelectric signals, such as electroencephalograph (EEG) signals, from a subject generally should address various requirements including safety needs, cost, power consumption, performance, ease of use and subject comfort. In a non-clinical application the relative importance of these factors may be somewhat different to that in a clinical application. For example, in a clinical application the electrodes are applied by a relatively skilled technician, whereas in non-clinical application the electrodes are more likely to be applied by a person with no training or knowledge of correct application or placement of the electrodes. Convenience and subject comfort are also generally more important in a non-clinical application. A patient in a clinical situation is more likely to be tolerant of some level of discomfort or inconvenience when testing and calibrating electrodes than a person in a non-clinical setting.

Conventional electrodes include passive electrodes and active electrodes. Passive electrodes follow a simple design principle and include a metal disc with a connecting wire to electronic circuitry. The simplicity makes this type of electrode low cost, although these electrodes are prone to noise and can require numerous noise canceling techniques to achieve satisfactory performance. One noise canceling technique, to minimize impedance at the skin-electrode interface and to minimize interference, involves conditioning the skin where the electrode is to be applied. Typically a scalpel is used to scrape the skin and a liquid disinfectant solution is used to clean the area. Another approach to minimizing impedance and interference at the skin-electrode interface, commonly combined with abrasive and depilatory preparation, is to fill any gap at the interface with a conductive gel or saline solution that can regulate the impedance. In this case, which is typical of biopotential electrodes, the conduction of signals is enhanced by the use of an electrolyte that matches the lowest-impedance internal conductive mode of the body. Where an electrolyte, such as saline or conductive gel is used, an electrical connection to the circuit is most commonly maintained with carefully chosen electrode plate materials that interface with the electrolyte. Typically silver/silver chloride is the material of choice for this contact due to its electrolytic properties including non-polarizability and rapid settling time. Silver/silver chloride electrodes cannot be used in direct long-term contact with the skin due to the cumulative toxicity of silver, therefore an interposing electrolyte material is required.

A conventional apparatus for applying electrodes to a subject's head includes a flexible cap that covers the subject's entire scalp and includes a strap beneath the chin, so that the cap may be snugly secured to the subject's head. This type of apparatus is typically used in a clinical setting and can include over 100 electrodes for some applications.

### SUMMARY

In general, in one aspect, the invention features an apparatus including a, conductive element formed from a non-adhesive hydrogel material. The conductive element is configured to provide a conductive path between an electrode and a subject's skin for transmitting EEG signals from the subject to the electrode.

Implementations of THE apparatus can include one or more of the following features. The hydrogel material can be selected such that the conductive element retains a desired shape and configuration after more than one use. The hydrogel material can be selected such that the conductive element can be hydrated and re-used repeatedly. The conductive element can be configured to fit around at least a portion of the electrode. The hydrogel material can be selected such that the conductive element is flexible and conforms to the subject's skin under a compressive force and maintains structural integrity for repeated use. The apparatus can further include a housing configured to house at least a portion of the conductive element. The housing can be formed from a material to resist drying of the conductive element. In one implementation, the housing is configured to facilitate penetration of a hair layer on the subject's scalp.

In general, in another aspect, the invention features an apparatus including a conductive element formed from a non-adhesive hydrogel material. The conductive element is configured to penetrate a hair layer above a subject's scalp to provide a conductive path from the subject's skin to an electrode in contact with the conductive element.

Implementations of the apparatus can include one or more of the following features. The hydrogel material can be selected such that the conductive element retains a desired shape and configuration after more than one use. The hydrogel material can be selected such that the conductive element can be hydrated and re-used repeatedly. The conductive element can be configured to fit around at least a portion of the electrode. The hydrogel material can be selected such that the conductive element is flexible and conforms to the subject's skin under a compressive force and maintains structural integrity for repeated use.

In general, in another aspect, the invention features a conductive assembly including a housing, an electrode plate element and a conductive element. The housing includes a first opening on a distal surface, a second opening on a proximal surface and a cavity within the housing. The electrode plate element is positioned within the housing and includes a contact surface exposed through the second opening of the housing. The conductive element is formed from a non-adhesive hydrogel material and positioned about a distal portion of the electrode plate element. A distal end of the conductive element is exposed through the first opening of the housing and is configured to provide a conductive path from a subject's skin to the electrode plate element.

Implementations of the conductive assembly can include one or more of the following features. The conductive assembly can further include a sensor circuit electrically connected to the contact surface of the electrode plate element. The hydrogel material of the conductive element can be selected such that the conductive element retains a desired shape and configuration after more than one use. The hydrogel material can be selected such that the conductive element can be hydrated and re-used repeatedly. The hydrogel material can be selected such that the conductive element is flexible and conforms to the subject's skin under a compressive force and maintains structural integrity for repeated use.

In general, in another aspect, the invention features an apparatus including a conductive element and a housing. The conductive element is formed from a non-adhesive hydrogel material positioned at least partially within the housing. The housing includes a cavity to house the conductive element and an electrode plate. The housing further includes an opening from which a contact surface of the conductive element is exposed. The housing tapers from a base region to a region including the opening such that the housing is configured to penetrate a hair layer above a subject's scalp to expose the subject's skin to the contact surface of the conductive element providing a conductive path to the electrode plate.

Implementations of the apparatus can include one or more of the following features. The hydrogel material of the conductive element can be selected such that the conductive element retains a desired shape and configuration after more than one use. The hydrogel material can be selected such that the conductive element can be hydrated and re-used repeatedly. The hydrogel material can be selected such that the conductive element is flexible and conforms to the subject's skin under a compressive force and maintains structural integrity for repeated use.

In general, in another aspect, the invention features an apparatus including an electrode plate, a sensor circuit and a non-adhesive conductive element. The sensor circuit is electrically connected to the electrode plate. The non-adhesive conductive element is formed from a hydrogel material and includes a contact surface configured to contact a subject's skin. The conductive element contacts at least a portion of the electrode plate and provides a conductive path between the subject's skin and the electrode plate for transmitting EEG signals from the subject to the electrode plate.

Implementations of the apparatus can include one or more of the following features. The hydrogel material can be selected such that the conductive element retains a desired shape and configuration after more than one use. The hydrogel material can be selected such that the conductive element can be hydrated and re-used repeatedly. The conductive element can be configured to fit around at least a portion of the electrode plate. The hydrogel material can be selected such that the conductive element is flexible and conforms to the subject's skin under a compressive force and maintains structural integrity for repeated use. The apparatus can further include a printed circuit board (PCB), wherein the sensor circuit is formed on the PCB.

In general, in another aspect, the invention features an electrode assembly including a printed circuit board (PCB) contained within a substantially waterproof housing and an electrode plate. The housing includes a first aperture in a lower surface. The electrode plate is attached to a lower surface of a base. An upper surface of the base is configured to attach to the housing containing the PCB. The base includes a second aperture aligned with the first aperture included in the lower surface of the housing. A conductive material is positioned within the first and second apertures and in contact with the electrode plate and the PCB thereby providing an electrical connection therebetween. The electrode assembly further includes a conductive element formed from a non-adhesive hydrogel material including an upper surface in contact with the electrode plate and a lower surface configured to contact a subject's skin. The conductive element provides a conductive path from the subject's skin to the PCB by way of the electrode plate therebetween for transmitting EEG signals from the subject to the electrode plate.

Implementations of the electrode assembly can include one or more of the following features. The hydrogel material can be selected such that the conductive element retains a desired shape and configuration after more than one use. The hydrogel material can be selected such that the conductive element can be hydrated and re-used repeatedly. The hydrogel material can be selected such that the conductive element is flexible and conforms to the subject's skin under a compressive force and maintains structural integrity for repeated use.

In general, in another aspect, the invention features an electrode including an electrode plate, a sensor circuit, a gimbaled contact element and a conductive flexure element. The sensor circuit is electrically connected to the electrode plate. The gimbaled contact element is configured to contact a subject's scalp and includes a non-adhesive hydrogel material for transmitting EEG signals from the subject's scalp to the electrode plate. The conductive flexure element connects the electrode plate and the gimbaled contact element and provides a conductive path therebetween.

Implementations of the invention can include one or more of the following features. The hydrogel material can be selected such that the conductive element retains a desired shape and configuration after more than one use. The hydrogel material can be selected such that the conductive element can be hydrated and re-used repeatedly. The hydrogel material can be selected such that the conductive element is flexible and conforms to the subject's skin under a compressive force and maintains structural integrity for repeated use.

Implementations of the invention can realize one or more of the following advantages. The use of a dry or semi-dry material at an interface with a subject's body eliminates the necessity to prepare the skin and apply liquid saline, oil or water-based contact gel, which can be invasive and difficult for routine consumer use. Such skin preparation solutions generally have an undesirable feel and require inconvenient cleaning to remove. Further, skin preparation solutions have the potential to induce local irritation, particularly when used with abrasives or depilation, and possibly in the presence of other unknown skin or hair preparations that typically are not cleaned off in a consumer application. By contrast, using materials already approved for and used in direct contact with the surface of a human eye has the advantage of demonstrated non-irritant and hypoallergenic properties to a higher level than required for skin contact devices.

The use of a soft material simultaneously improves electrode contact impedance and user comfort by being soft and deformable. A larger contact surface area is achieved by the application of a slight force, decreasing electrical impedance but also reducing the overall applied skin pressure for the same applied contact force.

The use of an elastomeric polymer material conventionally used for contact lenses in an implementation where the material is hydrated with an electrolyte, e.g., saline or another ionic liquid, maintains an electrolytic contact desirable for skin contact electrodes, while isolating the silver ions included in the electrode away from the skin surface. This can provide near-ideal conduction and eliminate the risk of transfer of silver ions through conventional liquid or gel electrolyte phases.

The use of a contact-lens-grade material can realize a number of commercial advantages. The materials are readily available at a relatively low cost, due to the existing high-volume market for the material in the contact-lens realm. The materials are typically polymerized from liquid precursors and can therefore be cast into specific shapes using available molding technology. This can simplify manufacture and further reduce the cost of fabrication of desired shapes. The materials have pre-existing, known biocompatibility, product-safety approvals and manufacturing quality assurance systems appropriate to medical grade products. Maintenance and cleaning materials are also readily available for implementations of the hydrogel conductive element that can use saline or contact lens hydration fluids to repeatedly hydrate and clean the elements. The materials are extremely rugged, having been designed primarily for use in very thin dimensions. The materials have the potential to be re-used indefinitely, including when used in implementations of the hydrogel conductive element that use saline or contact lens hydration solutions for hydration. The materials are naturally transparent, but can also be tinted to different shades using safe, pre-approved coloring agents, allowing flexibility in product design and aesthetics.

As contrasted to hydrogel materials used to provide electrolytic contact for ECG electrodes used to monitor cardiac activity or to apply voltage pulses, the materials used in the hydrogel conductive elements described herein have structural integrity. The hydrogel materials used in the ECG context typically have a consistency of a soft gel that is adhesive to allow rapid placement onto relatively hairless body parts, such as the chest wall. In addition to their adhesive properties, they have no structural integrity and usually adhere to cloth-backed sheets. They cannot be formed into desired shapes that retain their integrity over time. Hair penetration ability is limited by the adhesive nature of the material. ECG hydrogel materials are designed to be disposable after a single use. By contrast, the hydrogel conductive elements described herein are soft but extremely durable, non-adhesive and can maintain their structural integrity. Structural designs can be realized that allow direct penetration through a hair layer and the elements can be used for an indefinite number of repeat applications.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective side view of an example electrode conductive element.

FIG. 2 is a cross-sectional view of the example electrode conductive element of FIG. 1 taken along line 2-2.

FIG. 3 is a schematic representation of an example signal acquisition system.

FIG. 4 is a schematic representation of a 10-20 electrode placement scheme.

FIG. 5 is a perspective view of an example electrode headset.

FIG. 6A is a perspective view of an example electrode mounting assembly.

FIG. 6B is a perspective view of an electrode mounted within the electrode mounting assembly shown in FIG. 6A.

FIGS. 7A-B show an example electrode.

FIGS. 8A-B show an alternative example electrode.

FIGS. 9A-B show an alternative example electrode.

FIG. 10 shows a schematic representation of an example circuit diagram.

Like reference symbols in the various drawings indicate like elements.

### DETAILED DESCRIPTION

Providing a conductive fluid between an electrode contact and a location on a subject from which a signal is to be sensed can provide a stronger signal to the electrode. However, depending on the application, using a conductive gel or other wet fluid can be impractical. Further, outside of a medical treatment context, a subject is less likely to agree to having hair removed from the location. A conductive element is described that can be used with an electrode to provide an improved signal from the subject to the electrode, without requiring the subject's hair to be removed and without the discomfort of a liquid gel or other fluid. In some implementations, the conductive element incorporates electrolytic contact. The conductive element can be formed of a non-adhesive material that is sufficiently flexible and compressible to conform to the subject's skin without discomfort when placed in contact with the subject. The material can be one that does not dissolve in water, but is highly water-permeable. For example, the conductive element can be formed of a network of water-insoluble polymer chains, e.g., a hydrogel. The conductive element is formed from a material that can be shaped into a desired configuration, depending on the application. In one example, the conductive element can be formed from a hydrophilic structural polymer.

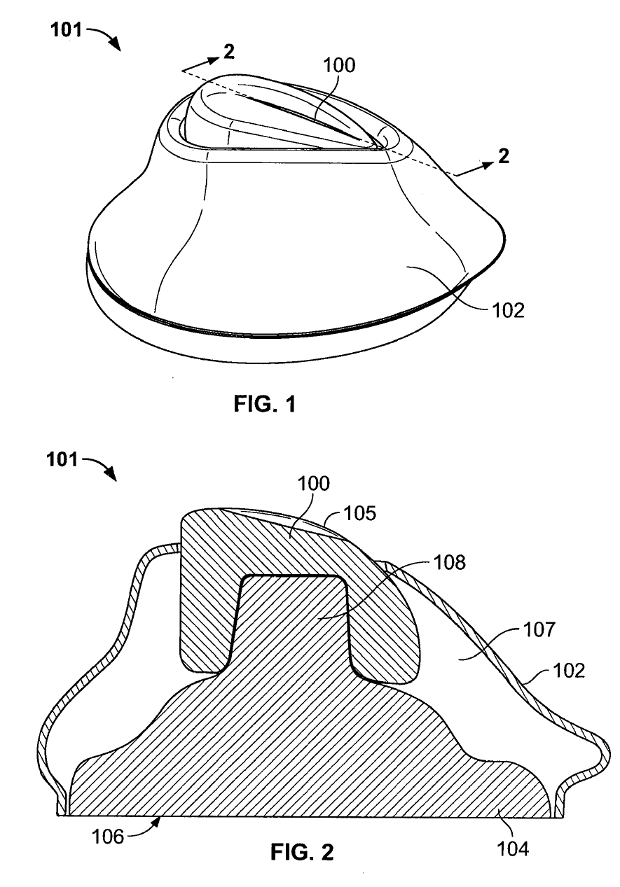

Referring to FIG. 1, a perspective side view is shown of an example conductive assembly 101 including a hydrogel conductive element 100 and a housing 102. In the particular implementation shown, the conductive element 100 has an approximate teardrop-shaped horizontal cross-section. The shape of the conductive element 100 can facilitate contact with a subject's skin while penetrating through the subject's hair, for example, if used with an electrode to sense EEG signals from the subject's scalp.

Referring to FIG. 2, a cross-sectional side view of the example hydrogel conductive element 100 and housing 102 is shown. The housing 102 can be formed from a non-conductive material that is water-impermeable, durable and biocompatible for skin contact. Example materials include polypropylene, polyethylene, nylon and polystyrene. The housing includes thin walls forming an inner cavity 107. Within the inner cavity 107 is housed an electrode plate 104.

In this particular example, the electrode plate 104 includes a nipple-shaped upper portion 108. The hydrogel conductive element 102 is shaped to fit over the upper portion 108, as shown in the cross-sectional view. In this example, the conductive element 102 fits snugly over the upper portion 108. The exterior surface 105 of the hydrogel conductive element contacts a subject's skin and effects an electrolytic connection to the electrode plate 104. A lower surface 106 of the electrode plate 104 is configured to mate with an electrical contact on a mounting apparatus within which the assembly 200 can be mounted. In some implementations, the electrode plate 104 is formed from silver/silver chloride. The electrical contact on the mounting apparatus is electrically connected to circuitry to receive signals from the electrode plate 104. The circuitry can be external to the mounting apparatus, or embedded therein, as is described further below for some particular implementations. Any suitable electrode can be used together with the conductive element, and some different example electrodes are discussed below.

In the implementations shown in FIGS. 1 and 2, the housing is configured to facilitate penetrating a hair layer. That is, the housing tapers from its base toward the distal region that makes contact with the subject's scalp. Having a narrow, tapered distal region operates to part the subject's hair and improve scalp contact. Other housing configurations can be used, depending on the particular implementation.

The conductive element 100 is formed by shaping a hydrogel material into the desired configuration. Examples of hydrogel materials include, but are not limited to: poly-hydroxyethylmethylacrylic (polyHEMA) materials, super-absorbent polymers such as poly-methacrylic acid, hygroscopic materials such as silica gel and porous silicones. The silicone and polyHEMA materials have the following mechanical and electrical properties that are desirable for use in data collection, e.g., EEG data collection: soft, pliable, durable, good set-up times, good conductivity and high quality signals. The material used to form the conductive element 100 is non-adhesive and can exhibit structural integrity. That is, once formed into a desired shape and configuration, subject to deformation under compression, the conductive element 100 substantially retains the shape and configuration.

In one implementation, the conductive element is formed from a material currently used to make disposable contact lenses, for example, polyBEMA with a nominal water content in excess of approximately 20% and saturated in a hydration fluid, such as saline. These materials are particularly rugged mechanically, soft and pliable when hydrated and are able to provide a suitable level of signal quality. Further, these materials are capable of being cast, molded or machined into desired shapes, allowing design flexibility. For example, in EEG application requiring data collection on a subject's scalp, the material can be configured into a shape that can penetrate the hair layer. The material, being soft and compressible, allows the conductive element to conform to a local skin profile and contact skin around remnant hairs. The conductive element is also comfortable and cushions the subject's scalp from the force required to maintain electrical contact.

In other implementations, the conductive element 100 can be formed into a different shape, depending on the configuration of the electrode plate and the external circuit type with which it will be used and/or the application. The particular configuration shown in FIGS. 1 and 2 is but one illustrative example. Of importance though is that the hydrogel material used can be configured into a shape that will maintain its structural integrity. Further, in addition to providing a conductive path for signals from the subject to the electrode plate, the shape itself can perform other functions, e.g., to penetrate the hair layer.

Advantageously, using materials that are already in use for contact lenses provides a material that is hypoallergenic and approved for biological use in worldwide markets. The materials are currently manufactured in medically controlled environments, and approved hydration materials with benign antibacterial and anti-mold properties are freely available and inexpensive.

In some implementations, the conductive element 100 can be repeatedly hydrated by soaking the conductive element 100 in a custom hydration solution or in a commonly available saline solution suitable for contact lenses. The conductive element 100 can be hydrated in situ within the assembly 101 or can be removed, hydrated and replaced within the assembly 101.

### Example Bio-Sensing System

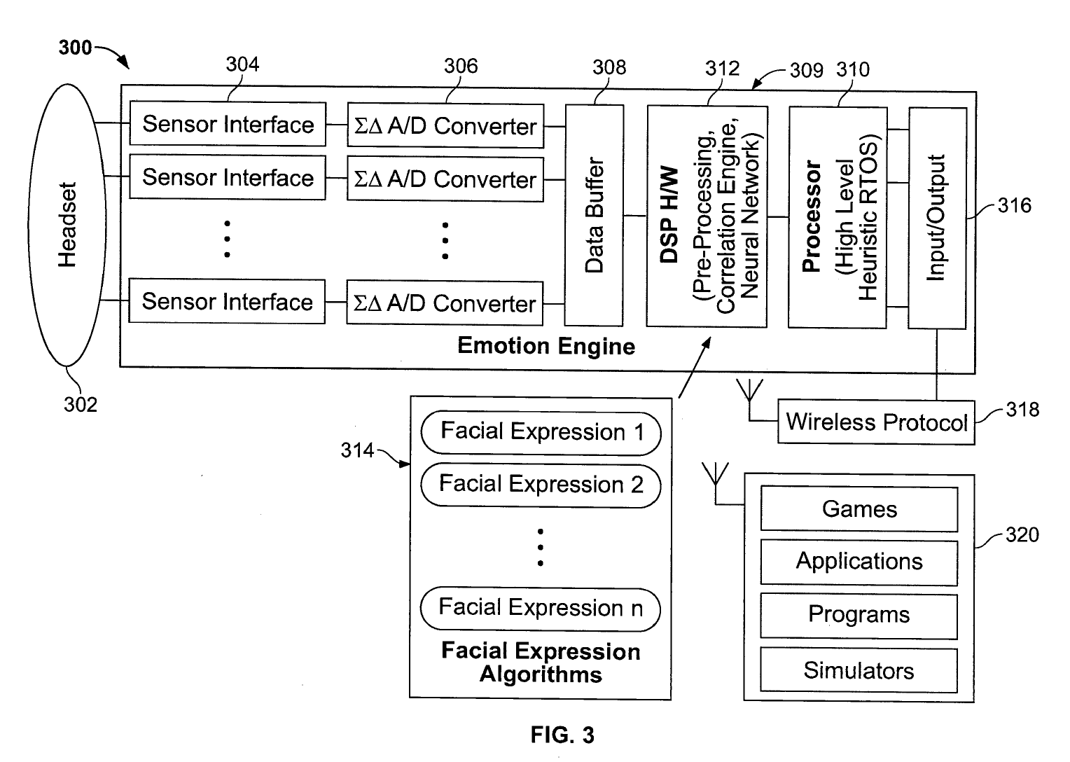

FIG. 3 is a schematic representation of a system for detecting and classifying mental states. The system is one example of a system that can employ an electrode using the conductive element described herein. It should be understood however that other systems can use an electrode used with the conductive element described, and the system shown in FIG. 3 is but one implementation for illustrative purposes.

The system includes a headset 302 configured to position one or more electrodes on a subject's head. In one implementation, the one or more electrodes include signal acquisition electrodes configured to detect signals such as electroencephalograph (EEG) signals, electro-oculograph (EOG) signals, or similar electrical potentials in the body. Signals detected by the electrodes in the headset 302 are fed through a sensor interface 304 and digitized by an analog to digital converter 306. Digitized samples of the signal captured by each of the electrodes can be stored during operation of the system 300 in a data buffer 308 for subsequent processing. In other implementations, the digitized samples of the signal can be transmitted over a wired or wireless connection to a distributed computer system for algorithmic processing. Such transmission links and distribution of computing resources can be transparent to the process described herein. By way of illustrative examples, the distributed computer system can be a gaming console or a custom processor.

The system 300 further includes a processing system 309 including a digital signal processor 312, a co-processing device 310 and associated memory for storing a series of instructions, otherwise known as a computer program or computer control logic, to cause the processing system 309 to perform desired functional steps. Notably, the memory includes a series of instructions defining at least one algorithm 314 for detecting and classifying a predetermined type of mental state. Mental states determined by such a classification can include, but are not limited to: an emotion; a desire, an intention or conscious effort to perform an action such as performing an interaction with a real or virtual object; and a mental state corresponding to an actual movement made by the subject, such as a facial expression, blink, gesture etc. Upon detection of each predefined type of mental state, a corresponding control signal is transmitted to an input/output interface 316. From the input/output interface, the control sign can be transmitted via a wireless transmission device 318 or a wired link (not shown) to a platform 320 for use as a control input by a gaming application, program, simulator or other application.

In this embodiment, the processing of signals, e.g. the detection or classification of mental states is performed in software and the series of instructions is stored in the memory. In another embodiment, signal processing can be implemented primarily in hardware using, for example, hardware components such as an Application Specific Integrated Circuit (ASIC). Implementation of the hardware state machine so as to perform these functions will be apparent to persons skilled in the relevant art. In yet other embodiments signal processing can be implemented using a combination of both software and hardware.

In this embodiment the processing system 309 is arranged as separate to the platform 320, however the system 300 can be arranged in a variety of configurations that split the signal processing functionality between various groups of hardware, for example in some embodiments, at least part of the signal processing functionality can be implemented in electronics mounted on the headset 302 or in the platform 320. For example, the apparatus can include a headset assembly that includes the headset, a MUX, A/D converter(s) before or after the MUX, a wireless transmission device, a battery for power supply, and a microcontroller to control battery use, send data from the MUX or A/D converter to the wireless chip, and the like. The apparatus can also include a separate processor unit that includes a wireless receiver to receive data from the headset assembly, and the processing system, e.g., the digital signal processor and the co-processor. The processor unit can be connected to the platform by a wired or wireless connection. As another example, the apparatus can include a head set assembly as described above, the platform can include a wireless receiver to receive data from the headset assembly, and a digital signal processor dedicated to detection of mental states can be integrated directly into the platform. As yet another example, the apparatus can include a head set assembly as described above, the platform can include a wireless receiver to receive data from the headset assembly, and the mental state detection algorithms are performed in the platform by the same processor, e.g., a general purpose digital processor, that executes the application, programs, simulators or the like.

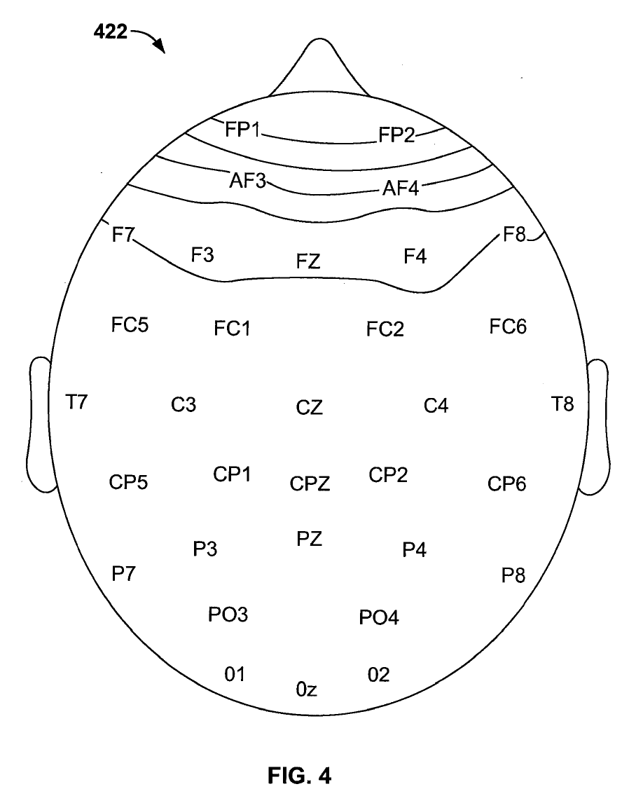

FIG. 4 shows a scheme 422 of electrode placement corresponding to the international 10-20 electrode placement system (the “10-20 system”). The 10-20 system is based on the relationship between the location of an electrode and the underlying area of cerebral cortex. Each point on the electrode placement scheme 422 indicates a possible scalp electrode position. Each position is indicated by a letter to identify a brain lobe and a number or other letter to identify a hemisphere location. The letters F, T, C, P, and O stand for the frontal, temporal, central, parietal and occipital lobes of the brain. Even numbers refer to the right hemisphere and odd numbers refer to the left hemisphere. The letter Z refers to an electrode placed on the mid-line. The mid-line is a line along the scalp on the sagittal plane originating at the nasion and ending at the inion at the back of the head. The “10” and “20” refer to percentages of the mid-line division. The mid-line is divided into 7 positions, namely, Nasion, Fpz, Fz, Cz, Pz, Oz and Inion, and the angular intervals between adjacent positions are set at 10%, 20%, 20%, 20%, 20% and 10% of the mid-line length respectively.

### Example Rigid Electrode Headset

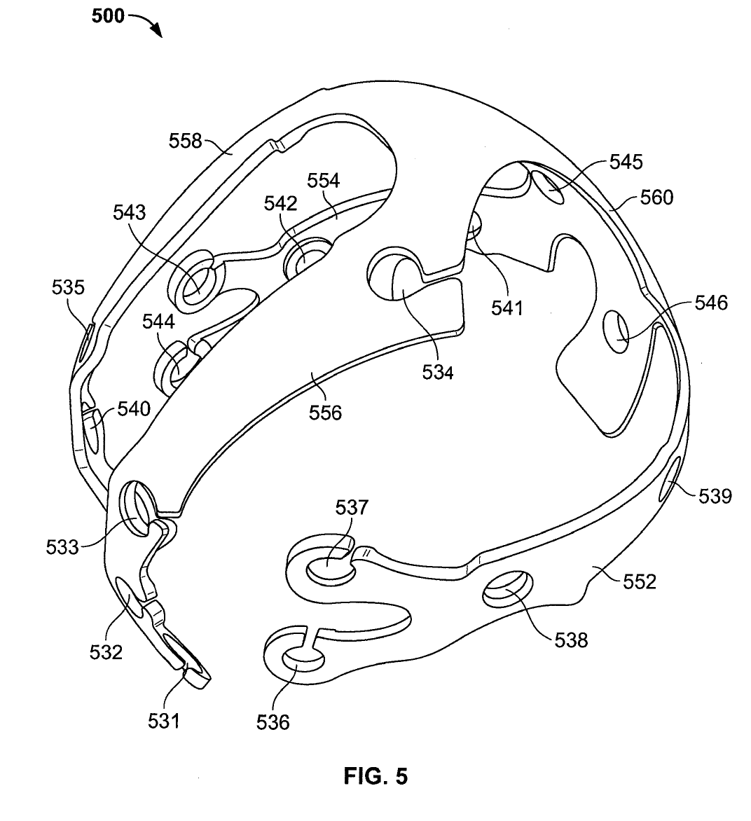

Referring to FIG. 5, one implementation of an electrode headset 500 is shown. The electrode headset 500 is configured to fit snugly on a subject's head and can properly fit a range of head shapes and sizes. Multiple electrode mounts are included in the electrode headset 500 and are each configured to mount an electrode. In this implementation the electrode mounts are apertures configured to receive and mount an electrode therein, and shall be referred to as electrode apertures 531-549. However, it should be noted that other configurations of electrode mounts can be used. For example, an electrode can be mounted to the electrode headset using a clamp, screw or other suitable connection mechanism and/or configuration.

In the particular implementation of the electrode headset 500 shown, the electrode apertures 531-549 are positioned to mount electrodes to gather information about the subject's facial expression (i.e., facial muscle movement), emotions and cognitive information. The electrode headset 500 can be used with electrodes mounted in all or a subset of the electrode apertures (531-546 are shown; some are not visible in this view). One or more apertures can be used to mount a reference electrode, i.e., an electrode to which signals received from other electrodes can be compared. In one implementation, the reference electrode can bias the subject's body to a known reference potential, e.g., one half of the analog supply voltage. Driven Right Leg (DRL) circuitry can compensate for external effects and keep the subject's body potential stable relative to the detection electronics. The EEG signals can be referenced to the body potential supped by the reference electrode.

The electrode headset 500 includes a left temporal band 552, a right temporal band 554, a left dorsal band 556 and a right dorsal band 558. The bands 552-558 each connect to a center band 560. Each band is configured to provide one or more electrode apertures to a desired region on a subject's head when the electrode headset 500 is worn by the subject. Generally, to provide desired results a particular electrode must be placed within a region that is approximately twice the size of the target location, providing at least some leeway when positioning the electrode on the subject's head. Because some leeway is permissible, and because the electrode headset 500 is configured to conform to and embrace heads of various shapes and sizes, the electrode headset 500 can be used to accurately position in accordance with a desired electrode placement scheme a set of electrodes on a variety of head shapes with relative ease of use.

The snug fit between the temporal bands 552, 554, that is provided at least in part by the bands 552, 554 pressing against the subject's head in an effort to return to their base position, exerts sufficient pressure on the electrodes mounted within the electrode apertures 536-539 and 541-544 to provide contact at the electrode-subject interface suitable to obtain a sufficient signal.

In one implementation, the center band 560 can be used to either house or mount electronic circuitry that is electrically connected to the one or more electrodes mounted within the electrode headset 500. The electronic circuitry can be configured to receive signals from the electrodes and provide an output to a processor and/or may be configured to perform at least some processing of the signals. For example, referring again to FIG. 3, in some implementations electronic circuitry mounted on or housed within the electrode headset 500 can be configured to perform some or all of the functions of the sensor interface 304, A/D converter 306, data buffer 308, processing system 309 and/or platform 320.

In one implementation, the electrode headset 500 is substantially formed from a polystyrene material, although other materials can be used including nylon. Optionally, some regions of the electrode headset 500 can be reinforced with an additional layer or extra thickness of the same or a different material, for example, a polystyrene reinforcement layer. Optionally, pads can be included in some regions such that the pads make contact with the subject's head and resist slippage against the subject's head and/or to improve the fit and subject's comfort. In one implementation the pads are formed from silicon.

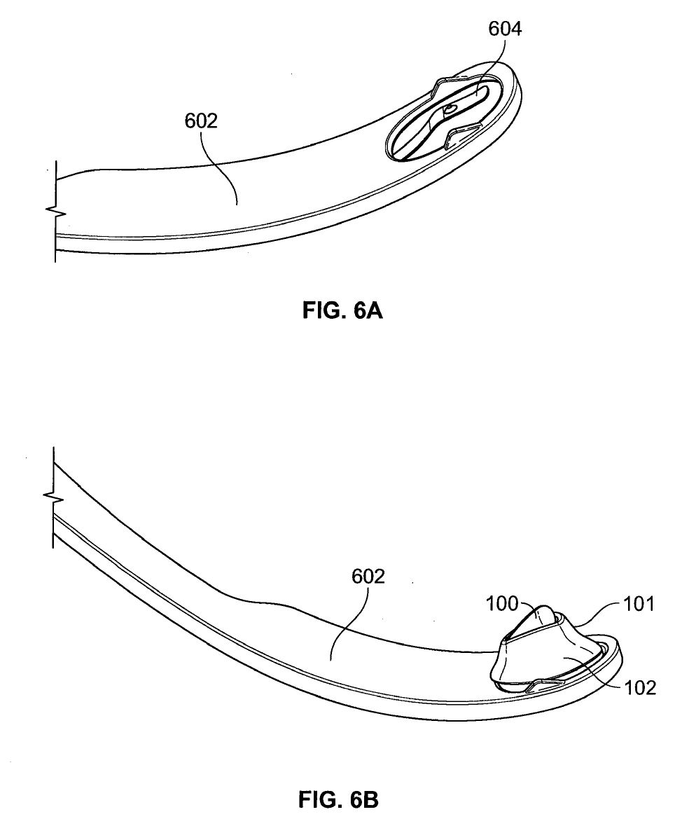

Referring now to FIGS. 6A and 6B, another implementation of an electrode mount that can be used in the electrode headset 500 or in another configuration of electrode headset or application, is shown. In this implementation, wires extending from the electrode mount 600 to electronic circuitry mounted on or housed within the electrode headset are embedded within an arm 602 included in the electrode headset 600. A flexible contact element 604 is exposed within the electrode mount 600, as shown in FIG. 6A.

The electrode mount 600 is configured to provide a snap fit connection to an electrode assembly 606 including a conductive assembly 101 formed similar to the one shown in FIGS. 1-2. The contact surface 106 of the electrode plate 104 housed within the conductive assembly 101 makes electrical contact with the flexible contact element 604 when the conductive assembly 101 is mounted therein, as shown in FIG. 6B. That is, the contact surface 106 of the electrode plate 104 electrically couples to the flexible contact element 604 included within the electrode mount 600, thereby electrically connecting the conductive assembly 101 to the electronic circuitry. The conductive element 100 provides a conductive path for signals from the subject's skin to the electrode plate 104. Other configurations of electrode mount can be used, and the one described is but one example.

### Example Electrodes

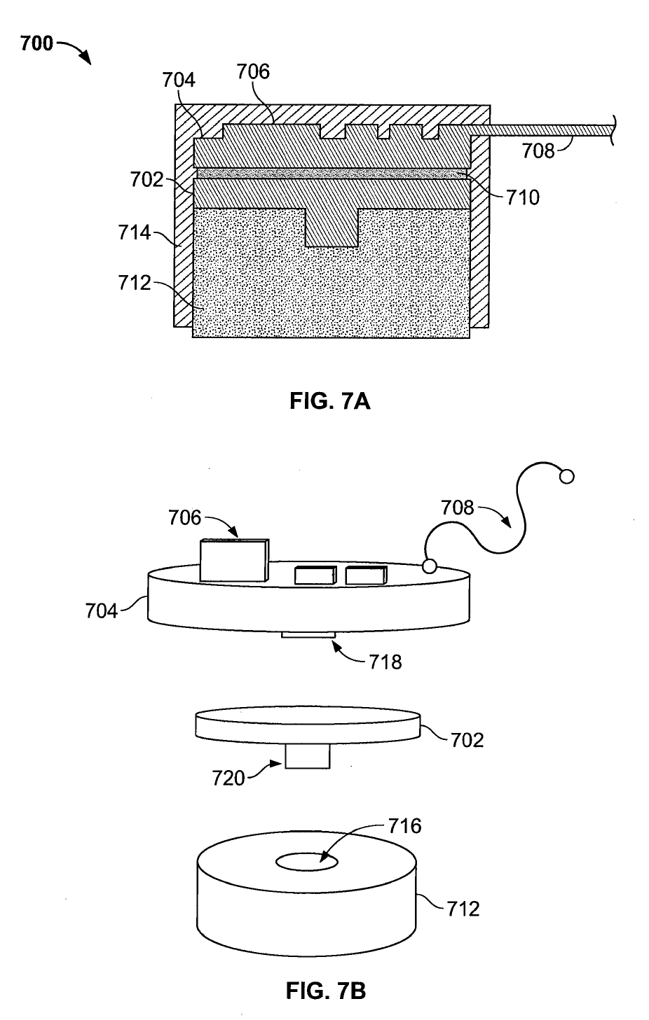

Referring to FIG. 7A, a schematic cross sectional view of one implementation of an electrode that can be used with the conductive element described herein is shown. The electrode can be used in the electrode headset described above, or in another type of mounting structure for the same or a different application. The electrode assembly 700 includes an electrode plate 702 mounted to a printed circuit board (PCB) 704. The PCB 704 includes electronic circuit components forming a sensor circuit. (denoted generally as 706). One or more wires 708 are connected to the sensor circuit 706 to provide power to the circuit 706 and permit signals to be sent to a signal acquisition system. The circuit 706 of the PCB 704 includes at least one electrical contact (not shown) that is configured to be connected to an electrode.

The electrode can be used to pick up bioelectrical potentials from the skin of a subject, and includes the electrode plate 702. The electrode plate 702 is maintained in electrical contact with at least one contact mounted on the PCB via a conductive medium, for example, a conductive glue 710. On the underside of the electrode plate 702 is mounted a hydrogel conductive element 712, which is configured to provide a conductive path between the subject's skin and the electrode plate 702 when in use. In this implementation, the conductive element 712 is shaped as a circular disk. However, in another implementation, the conductive element 712 can be shaped differently. For example, the conductive element 712 can be formed similar to the conductive element 100 shown in FIGS. IA-B, including an inner cavity that is formed to house at least a portion of the electrode assembly 700.

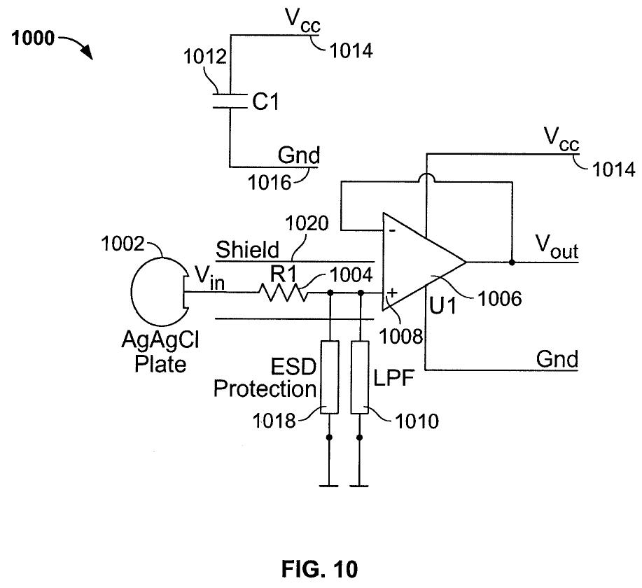

FIG. 7B illustrates a schematic exploded view of the PCB 704, electrode plate 702 and conductive element 712 shown in FIG. 7A. A circuit 706 as depicted in FIG. 10 is formed on the PCB 704. On the underside (or other convenient location) of the PCB 704 is a conductive contact 718. The conductive contact 718 can be made of copper or another suitably conductive material, and is used to make electrical contact between the sensor circuit 706 mounted on the PCB 704 and the electrode plate 702. One embodiment of the electrode plate 702 is made of silver-silver chloride (AgAgCl) and is generally disk-like in shape. An upper surface of the electrode plate 702 is maintained in electrical contact with the contact 718, either directly or via a conductive material such as a silver epoxy conductive glue. The bottom surface of the electrode plate 702 makes contact with the conductive element 712.

On the underside of the electrode plate 702 is a generally cylindrical projection 720. The projection 720 is configured to be received into a correspondingly shaped recess 716 formed in the upper side of the conductive element 712. The protrusion 720 is sized to as to be a friction fit with the receiving hole 716 in the conductive element 712, and to thereby provide a secure mounting arrangement for fixing the conductive element 712. The projection 720 also increases the amount of surface area of the electrode plate 702 that makes contact with the conductive element 712, and therefore can increase the quality of signal acquisition. However, in alternative embodiments the mating surfaces of the electrode plate 702 and conductive element 712 can be flat, or can have an alternative shape or can be attached together differently.

In use the conductive element 712 can absorb and hold electrolytic solution such as saline solution or other electrically conductive liquid and maintain a flexible and high quality conductive link between the subject's skin and the electrode plate 702. In order to protect the electronics of the electrode assembly from damage and to improve the safety of the electrode, the PCB can be enclosed in a waterproof housing. The waterproofed PCB and the attached electrode plate arrangement can be inserted into the housing 714.

In some embodiments, the electrode casing includes a plastic component of unitary construction. The casing can be tubular in configuration and serve a dual role of ensuring mechanical strength of the electrode arrangement and have an open end that can serve as a feed tube, through which electrolyte solution can be introduced to the conductive element 712. The inside of the recess into which the PCB-electrode arrangement is received can include one or more retaining formations configured to hold the PCB-electrode arrangement and conductive element in place during use. The assembly can include a closure or other means to secure the PCB-electrode arrangement in the housing. Moreover, in one embodiment the housing 714 can be configured to hold the PCB-electrode arrangement in a releasable manner to facilitate replacement of the PCB-electrode arrangement within the housing. The inside of the housing 714 can be provided with teeth or circumferential ribs to hold the PCB-electrode arrangement in place, and allow the PCB-electrode arrangement to be pushed out for replacement. The replacement process requires connecting the replacement PCB-electrode arrangement into the acquisition system. In one implementation, this can be achieved using a known crimping or modular wiring/connector systems.

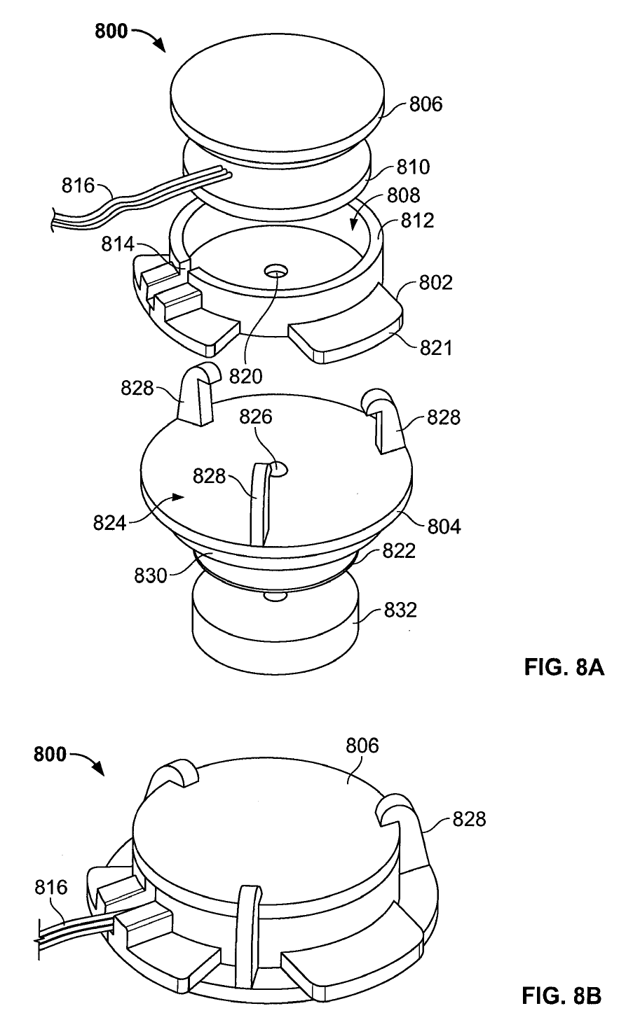

Referring to FIGS. 8A-B, an alternative electrode assembly is shown that can be used with a hydrogel conductive element as described herein. This electrode assembly can also be used in an electrode headset as described above, or in a different mounting structure for the same or a different application. The electrode assembly 800 of this embodiment includes a PCB receiving portion 802, a base portion 804 and a cap 806. The PCB receiving portion 802 includes a cavity 808 and can be waterproofed, using a material that can also be used to hold the PCB 810 in place in the housing. An opening 814 allows wires 816 to extend to the PCB 810. The floor 818 of the cavity 808 is provided with an aperture 820 to enable an electrical connection to be made between an electrode circuit on the PCB 810 and an electrode plate 822. The PCB receiving portion 802 also includes one or more radial projections 821, described further below.

A cap 806 is provided that is configured so as to close off the cavity 808 and hold the PCB 810 in place within the housing. The base 804 is mounted below the PCB receiving portion 802, and includes a base portion 824 with a through hole 826. The through hole 826 is provided to enable an electrical connection to be made, through the base 804, between a contact of the electrode circuit on the PCB 810 and an electrode plate 822.

The base 804 also includes a plurality (three in this embodiment) of retaining members 828 that, when the housing is assembled, clip over the edge of the cap 806 and retain the cap 806 in place. The underside of the base 804 further includes an annular flange 830, that defines a recess into which the electrode plate 822 is mounted. The electrode plate 822 can be attached to the bottom of the base 804 using, for example, a conductive glue. In use, sufficient glue is used to mount the electrode plate 822 to the base 804 such that the voids formed by the through holes 826 and 820 are substantially filled and electrical contact is made with a contact of the electrode circuit on the PCB 810.

A hydrogel conductive element 832 is mounted on the electrode. In this implementation, the conductive element 832 is shaped as a circular disk. However, in another implementation, the conductive element 832 can be shaped differently. For example, the conductive element 832 can be formed similar to the conductive element 100 shown in FIGS. 1A-B, including an inner cavity that is formed to house the electrode assembly 800. The conductive element 832 provides a conductive path from the electrode plate 822 to the skin of the subject.

FIG. 8B depicts the electrode assembly of FIG. 8A in an assembled state. The electrode housing components can be made from a plastic material such as polyurethane. Such components can be made using from RTV molds created from fabricated styrene masters or by injection molding. Moreover in these embodiments the housings can have one or more electrolyte feed ducts that bypass non-waterproofed electronic components (or be configured to receive an external tube) that can enable electrolyte fluid to be applied to the contact pad of the electrode assembly in use. Such ducts can preferably allow application of the electrolyte fluid without removal of the electrodes from the subject.

It should be noted that since, electrode assemblies can be expensive it is advantageous to enable the number or electrodes to be increased and decreased by the manufacturer or subject to suit his or her needs. For example, an electrode headset in a certain application, e.g., detecting an emotion, may only need eight electrodes, whilst for another application, e.g., additionally detecting a conscious effort such as to move a real or virtual object, or a muscle movement, one or more additional electrodes may be needed. Therefore the electrodes should be mountable and detachable from the headset, e.g., electrode headset 500 for example, in the manner discussed above.

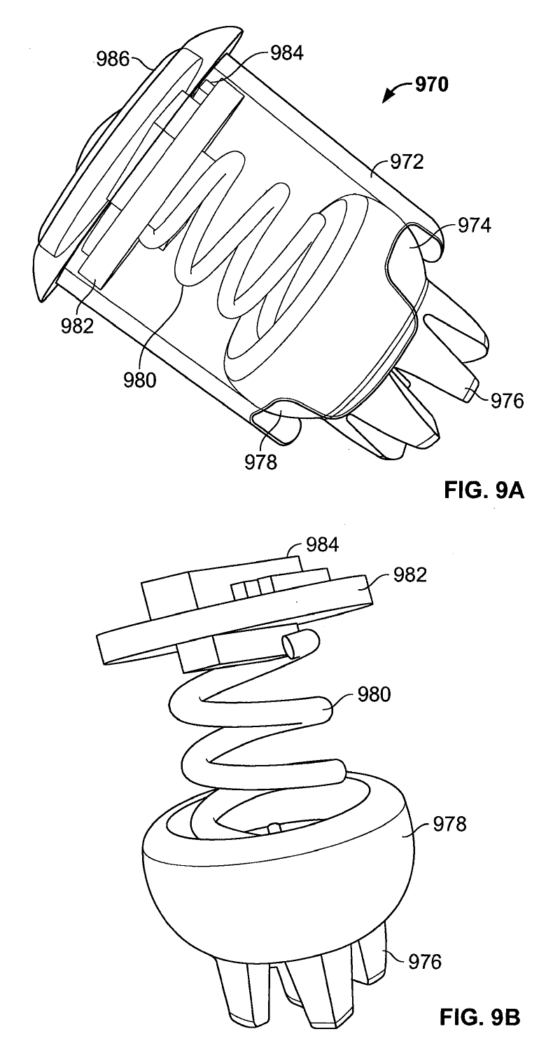

Referring to FIGS. 9A-B, another implementation of an electrode 970 that can be used with the hydrogel conductive element described herein is shown. The electrode 970 can be mounted within the electrode headset 500 described above, or used independent of the electrode headset 500 for a different application. In this implementation, the electrode 970 is configured as an active resistive electrode. The electrode includes a housing 972, which for illustrative purposes is shown as transparent, including a substantially tubular body 972 and a cap 986. Referring particularly to FIG. 9B, the electrode 970 is shown with the housing 972 removed for illustrative purposes. The electrode 970 includes a printed circuit board (PCB) 984 attached to an electrode plate 982. The PCB 984 includes electronic circuit components forming a sensor circuit. One or more wires can connect to the sensor circuit to provide power to the circuit and permit signals to be sent from the sensor circuit to a signal acquisition system, which can be mounted or housed within the electrode headset 500 or located external to the electrode headset 500.

A flexure element 980 is attached to the underside of the electrode plate 982 and connects on a second end to a gimbaled contact 974. In this implementation the flexure element 980 is a spring, although in other implementations the flexure element can be configured differently. The gimbaled contact 974 includes an upper portion 978 forming a gimbaled connection to the housing 972. A lower portion of the gimbaled contact provides one or more hydrogel conductive elements 976 configured to contact the subject's skin. The flexure element 980 is formed from a conductive material, thereby electrically connecting the gimbaled contact 974 to the electrode plate 982. A conductive path is thereby provided from the subject's skin to the electrode plate 982 via the gimbaled contact 974, including the conductive elements 976, and flexure element 980. Bioelectrical potentials from the subject's skin detected by the gimbaled contact 974 are thereby provided to the electrode plate 982 and ultimately to the sensor circuit included in the PCB 984.

In some implementations, the hydrogel conductive elements 976 can be formed by casting into the desired shape, or by machining after polymerization when the material is a very hard solid.

The flexure element 980 can be made from a conductive material, for example, a metal. The electrode plate 982 can be made from biocompatible metal or biocompatible metal alloy and in one implementation is formed from silver-silver-chloride (AgAgCl). The electrode plate 982 material selection is important to ensure proper biosignal acquisition and minimize skin-electrode noise. Other example materials include: silver, gold and tin, but are not limited to these.

Various embodiments of the conductive elements 976 can be used. In an implementation where the electrodes will be used on a subject's head, preferably the conductive elements 976 are formed as elongated protrusions as shown, to provide sufficient contact with the subject's skin through the subject's hair.

In one implementation, the housing 972 is formed from plastic. The gimbaled contact, other than the conductive elements 976, can be formed from a biocompatible conductive material, for example, metal.

Referring now to FIGS. 5 and 9A, in one implementation, the tubular body 971 of the electrode is configured to friction fit within an electrode aperture included in the electrode headset 500. As described above, the electrode apertures can include an annular member that facilitates a friction fit to the outer surface of the tubular body 971. As previously described, each electrode 970 can be independently mounted within and removed from the electrode headset 500, allowing different subsets of electrodes to be used and allowing malfunctioning or broken electrodes 970 to be replaced.

An electrode headset 500 configured to receive an electrode 970 having the dimensions described above can include electrode apertures having an inner diameter sized to friction fit the tubular body 971 of the electrode 970. The inner diameter of the electrode apertures can vary, depending on the electrode to be mounted therein. In one implementation, the electrode apertures can have different inner diameters relative to one another, for example, if different sizes or types of electrodes are intended to be mounted in the various different electrode apertures.

### Circuit Diagram

Referring now to FIG. 10, a schematic circuit diagram is shown for an embodiment of an active electrode for sensing bioelectric potentials. The circuit 1000 depicted is suitable for use with an electrode or electrode assembly such as those shown in FIGS. 7A-B, 8A-B and 9A-B. The circuit 1000 includes an electrode plate 1002, that is maintained in electrical contact (directly or via a conductive path) with the subject's skin. For example, the electrode plate 1002 can be the electrode plate 104 shown in FIG. 2, the electrode plate 702 of the electrode assembly 700 shown in FIGS. 7A-B, the electrode plate 822 of the electrode assembly 800 shown in FIGS. 8A-B, or the electrode plate 982 of the electrode 970 shown in FIGS. 9A-B.

The electrode plate 1002 provides an input voltage (Vin) that is initially applied to an input protection resistor R1 1004. The input resistor R1 1004 serves as overcurrent protection in case of electrode malfunction, and protects both the operational amplifier U1 1006 and the subject. In one embodiment, R1 1004 is a 5 kΩ resistor. The input resistor R1 1004 is connected to a positive terminal 1008 of the operational amplifier U1 1006. The operational amplifier U1 1006 can be set up in a buffer amplifier arrangement. In this example, the buffer amplifier has a gain of 1, however other gains can be used. The operational amplifier U1 1006 can be a CMOS operational amplifier, which provides a large input impedance, e.g., in the gigaohm range. The operational amplifier U1 1006 can have a lower output impedance than a passive electrode, and reduce hum caused by environmental interference, such as power line noise. The operational amplifier U1 1006 can have low intrinsic noise in the frequency range of 0.1 to 40 Hz, in order to enable accurate detection of weak EEG signals such as evoked potentials. The operational amplifier U1 1006 preferably has low drift and low offset voltage.

In one embodiment, the operational amplifier U1 1006 is a Texas Instruments operational amplifier model No. TLC2201. Alternatively a TLV2211 operational amplifier (also from Texas Instruments) can be used and may be advantageous, as it has a smaller footprint and lower current consumption. As will be known to those skilled in the art other types of operational amplifiers can be used, e.g., a OPA333 operational amplifier (also from Texas Instruments). The circuit 1000 includes an optional low pass filter (LPF) 1010, which can be used to filter out noise introduced by sources such as radio frequency interference and that can affect the quality of signals required by the electrodes. The circuit 1000 also includes optional electro static discharge (ESD) 1018 protection circuitry to protect the operational amplifier U1 1006 in case of electrostatic discharge. The circuit 1000 includes a bypass capacitor C1 1012 connected between the power supply signal Vcc 1014 of operational amplifier U1 1006 and to ground 1016 to decouple the power supply. An optional PCB shield 1020 can be included around an input trace.

### Signal Acquisition System

As described above, the electrode headset is configured to mount therein one or more electrodes. Each electrode is electrically connected to electronic circuitry that can be configured to receive signals from the electrodes and provide an output to a processor. The electronic circuitry also may be configured to perform at least some processing of the signals received from the electrodes. In some implementations electronic circuitry mounted on or housed within the electrode headset can be configured to perform some or all of the functions of the sensor interface 304, A/D converter 306, data buffer 308, processing system 309 and/or platform 320.

In one implementation, the electronic circuitry is mounted on the electrode headset and electrically connected to each electrode mounted therein by one or more wires extending between the electronic circuitry and each electrode. The wires can be either visible on the exterior or interior of the electrode headset, or can be formed within the electrode headset, for example, by molding within one or more plastic components forming the electrode headset. Preferably, the wiring system exhibits one or more of the following features: a low cost; termination at the electronic module with a connector; flexible and shapeable to fit the contour of the electrode headset; strain relief at the conductor terminations; non-breakable flexible wiring with strain relief, moldable in a rigid headset; noise immunity and having conductor resistance less than 100 ohms.

### Alternative Implementations

It should also be understood that the electrode circuit arrangement, electrodes and electrode headset arrangements described herein can be used in connection in a wide variety of applications outside the implementations described herein. For example the electrode headset arrangement described herein can be used with other known electrode arrangements. Moreover the electrode arrangements described herein can be used to detect other types of bioelectric potentials on parts of the body other than the head, e.g. ECG. The electrodes described herein can also be useful for non-human applications.

It will be understood that the subject matter disclosed in this specification extends to all alternative combinations of two or more of the individual features mentioned or evident from the text or drawings. All of these different combinations constitute various alternative aspects.

A number of embodiments of the invention have been described. Nevertheless, it will be understood that various modifications may be made without departing from the spirit and scope of the invention. Accordingly, other embodiments are within the scope of the following claims.

What is claimed is:

1. An apparatus comprising:
a conductive element formed from a non-adhesive hydrogel material and configured to provide a conductive path between an electrode and a subject's skin for transmitting EEG signals from the subject to the electrode.

2. The apparatus of claim 1, wherein the hydrogel material is selected such that the conductive element retains a desired shape and configuration after more than one use.

3. The apparatus of claim 1, wherein the hydrogel material is selected such that the conductive element can be hydrated and re-used repeatedly.

4. The apparatus of claim 1, wherein the conductive element is configured to fit around at least a portion of the electrode.

5. The apparatus of claim 1, wherein the hydrogel material is selected such that the conductive element is flexible and conforms to the subject's skin under a compressive force and maintains structural integrity for repeated use.

6. The apparatus of claim 1, further comprising:
a housing configured to house at least a portion of the conductive element, where the housing is formed from a material to resist drying of the conductive element.

7. The apparatus of claim 6, wherein the housing is configured to facilitate penetration of a hair layer on the subject's scalp.

8. An apparatus comprising:
a conductive element formed from a non-adhesive hydrogel material and configured to penetrate a hair layer above a subject's scalp to provide a conductive path from the subject's skin to an electrode in contact with the conductive element.

9. The apparatus of claim 8, wherein the hydrogel material is selected such that the conductive element retains a desired shape and configuration after more than one use.

10. The apparatus of claim 8, wherein the hydrogel material is selected such that the conductive element can be hydrated and re-used repeatedly.

11. The apparatus of claim 8, wherein the conductive element is configured to fit around at least a portion of the electrode.

12. The apparatus of claim 8, wherein the hydrogel material is selected such that the conductive element is flexible and conforms to the subject's skin under a compressive force and maintains structural integrity for repeated use.

13. A conductive assembly comprising:
a housing providing a first opening on a distal surface, a second opening on a proximal surface and a cavity within the housing;
an electrode plate element positioned within the housing and including a contact surface exposed through the second opening of the housing; and
a conductive element formed from a non-adhesive hydrogel material and positioned about a distal portion of the electrode plate element, where a distal end of the conductive element is exposed through the first opening of the housing and is configured to provide a conductive path from a subject's skin to the electrode plate element.

14. The conductive assembly of claim 13, further comprising:
a sensor circuit electrically connected to the contact surface of the electrode plate element.

15. The conductive assembly of claim 13, wherein the hydrogel material of the conductive element is selected such that the conductive element retains a desired shape and configuration after more than one use.

16. The conductive assembly of claim 13, wherein the hydrogel material is selected such that the conductive element can be hydrated and re-used repeatedly.

17. The conductive assembly of claim 13, wherein the hydrogel material is selected such that the conductive element is flexible and conforms to the subject's skin under a compressive force and maintains structural integrity for repeated use.

18. An apparatus comprising:
a conductive element formed from a non-adhesive hydrogel material positioned at least partially within a housing;
the housing including a cavity to house the conductive element and an electrode plate and an opening from which a contact surface of the conductive element is exposed, where the housing tapers from a base region to a region including the opening such that the housing is configured to penetrate a hair layer above a subject's scalp to expose the subject's skin to the contact surface of the conductive element providing a conductive path to the electrode plate.

19. The apparatus of claim 18, wherein the hydrogel material of the conductive element is selected such that the conductive element retains a desired shape and configuration after more than one use.

20. The apparatus of claim 18, wherein the hydrogel material is selected such that the conductive element can be hydrated and re-used repeatedly.

21. The apparatus of claim 18, wherein the hydrogel material is selected such that the conductive element is flexible and conforms to the subject's skin under a compressive force and maintains structural integrity for repeated use.

22. An apparatus comprising:
an electrode plate;
a sensor circuit electrically connected to the electrode plate;
a non-adhesive conductive element formed from a hydrogel material and including a contact surface configured to contact a subject's skin, where the conductive element contacts at least a portion of the electrode plate and provides a conductive path between the subject's skin and the electrode plate for transmitting EEG signals from the subject to the electrode plate.

23. The apparatus of claim 22, wherein the hydrogel material is selected such that the conductive element retains a desired shape and configuration after more than one use.

24. The apparatus of claim 22, wherein the hydrogel material is selected such that the conductive element can be hydrated and re-used repeatedly.

25. The apparatus of claim 22, wherein the conductive element is configured to fit around at least a portion of the electrode plate.

26. The apparatus of claim 22, wherein the hydrogel material is selected such that the conductive element is flexible and conforms to the subject's skin under a compressive force and maintains structural integrity for repeated use.

27. The apparatus of claim 22, further comprising:
a printed circuit board (PCB), wherein the sensor circuit is formed on the PCB.

28. An electrode assembly comprising:
a printed circuit board (PCB) contained within a substantially waterproof housing, the housing including a first aperture in a lower surface;
an electrode plate attached to a lower surface of a base, where an upper surface of the base is configured to attach to the housing containing the PCB and where the base includes a second aperture aligned with the first aperture included in the lower surface of the housing;
a conductive material positioned within the first and second apertures and in contact with the electrode plate and the PCB thereby providing an electrical connection therebetween; and
a conductive element formed from a non-adhesive hydrogel material including an upper surface in contact with the electrode plate and a lower surface configured to contact a subject's skin, wherein the conductive element provides a conductive path from the subject's skin to the PCB by way of the electrode plate therebetween for transmitting EEG signals from the subject to the electrode plate.

29. The electrode assembly of claim 28, wherein the hydrogel material is selected such that the conductive element retains a desired shape and configuration after more than one use.

30. The electrode assembly of claim 28, wherein the hydrogel material is selected such that the conductive element can be hydrated and re-used repeatedly.

31. The electrode assembly of claim 28, wherein the hydrogel material is selected such that the conductive element is flexible and conforms to the subject's skin under a compressive force and maintains structural integrity for repeated use.

32. An electrode comprising:
an electrode plate;
a sensor circuit electrically connected to the electrode plate;
a gimbaled contact element configured to contact a subject's scalp and comprising a non-adhesive hydrogel material for transmitting EEG signals from the subject's scalp to the electrode plate;
a conductive flexure element connecting the electrode plate and the gimbaled contact element and providing a conductive path therebetween.

33. The electrode of claim 32, wherein the hydrogel material is selected such that the conductive element retains a desired shape and configuration after more than one use.

34. The electrode of claim 32, wherein the hydrogel material is selected such that the conductive element can be hydrated and re-used repeatedly.

35. The electrode of claim 32, wherein the hydrogel material is selected such that the conductive element is flexible and conforms to the subject's skin under a compressive force and maintains structural integrity for repeated use.
